(19) United States
(12) Patent Application Publication
(10) Pub. No.: US 2004/0214259 A1
(43) Pub. Date: Oct. 28, 2004

(54) Methods of sampling microbial communities and apparatus therefore

(75) Inventors: Aaron D. Peacock, Oak Ridge, TN (US); Greg A. Davis, Loudon, TN (US); David C. White, Knoxville, TN (US)

Correspondence Address: Frank C. Eisenschenk, Ph.D., Saliwanchik, Lloyd & Saliwanchik, A Professional Association, 2421 N.W. 41st Street, Suite A-1, Gainesville, FL, 32606, US

(73) Assignee: The University of Tennessee Research Foundation, Knoxville, TN

(21) Appl. No.: 10/788,432

(22) Filed: Feb. 27, 2004

Related U.S. Application Data
(60) Provisional application No. 60/450,880, filed on Feb. 28, 2003.

(51) Int. Cl. C12Q 1/18

(52) U.S. Cl. 435/32

(57) ABSTRACT

The subject invention provides an apparatus and methods for microbial sampling of solids, liquids, and gases, and in performing in situ microcosm or down-well subsurface solid phase sampler tests.

### CROSS-REFERENCE TO RELATED APPLICATIONS

This application claims the benefit of application 60/450,880, filed on Feb. 28, 2003. This application is hereby incorporated by reference in its entirety, including all figures, formulae, references, photographs, and tables.

The subject invention was made with government support under a research project supported by Grant DE-FC02-96ER62278, from the Office of Biological and Environmental Research (OBER) of the Office of Science (SC), U.S. Department of Energy (DOE), Natural and Accelerated Bioremediation Research (NABIR) Program (Assessment Element). The government may have certain rights in this invention.

### BACKGROUND OF INVENTION

The present invention relates to the process of detection subsets of the microbial multispecies consortia colonizing solid phase samplers in the presence of locally concentrated stable labeled (non-radioactive) isotopes in substrates, pollutants or from natural abundance stable isotopes in nutrient sources detected in biomarkers whose isotope content is detected by isotope ratio mass spectrometry after appropriate purification.

A major problem in understanding the roles of multispecies microbial communities in important processes like bioremediation of pollutants, bioimmobilization of heavy metals and nuclides, sequestration of carbon dioxide, microbial influenced corrosion, generation of acid mine drainage, microbial biofouling, medical/dental/veterinary biofilm problems, rhizosphere pathophysiology, agriculturally important processes, food spoilage and other problems is that three factors must known. Specifically, what bacteria are present, what is their activity, and what is their genetic capability. Bacteria produce specialized, measurable, intercellular constituents (lipids and nucleic acids) that are involved in or code for specific metabolic activities, which vary in response to the environment. The presence of, or relative quantities of, various constituents are indicative of specific metabolic activities, and hence provide direct evidence of environmental conditions under which the organisms are growing in situ. This information can be used to show that certain bioprocesses are occurring, and/or a particular set of conditions that favor a certain outcome can be obtained. Viable bacteria can be detected as those with intact polar phospholipid membranes which are readily assessed quantitatively with analysis of phospholipids ester-linked fatty acids (PLFA) [1]. PLFA are metabolically labile with phospholipases rapidly forming diglycerides from the phospholipids and thus PLFA do not exist as molecular fossils and are quantitative measures of viable microbial biomass [2,3]. The community composition of multispecies microbial communities can be quantitatively discerned from the PLFA and lipid biomarkers in many cases [4,5]. More specific definition of community composition can be achieved by isolating DNA from the communities then amplifying specific portions of the genome with PCR. This is done by proper selection of primers and in environmental applications is usually focused on the genes for ribosomal RNA called rDNA [6] although genes for specific functions can be utilized [7]. The PCR amplicons are then separated by denaturing gel gradient electrophoresis (DGGE), prominent bands cut out, eluted and the amplicons sequenced. The sequences are then compared to a database and phylogenetic matching gives indications of the community diversity [8].

Most research has focused on how much (biomass) and who (community composition/phylogeny) when conducting characterizations. Indeed much progress has been made to date across many areas in regards to identifying the environments and bacteria that are capable of specific processes or the functional genes etc. involved in the process. However there is a crucial link in the process that is missing, all of the current techniques, be they nucleic acid based or other biomarkers, only imply activity. The lipid composition reflects both the genotypes and phenotypic responses at the specific microniches of the bacteria as well as providing specific biomass quantitation [9]. We have developed the unique capability of defining the in situ redox conditions by measuring the respiratory ubiquinones and menaquinone isoprenologues and plasmalogen content [10] Metabolic stresses in the microbial community are estimated from specific PLFA ratios [1], diglycerides, and the stimulation of unbalanced growth are reflected in poly β-hydroxyalkanoate (PHA) [11]. These are useful and quantitative indicators of metabolic activity but currently there is no robust way available to directly and quantitatively measure microbial activity at the cellular level in situ. DNA and some lipid biomarkers provide evidence of what is potentially present, and functional gene doses could indicate an active process, but these are not definitive evidence for metabolic activity. Large portions of the sedimentary, groundwater or biofilm communities might be present with the correct genetic potential, potentially viable, but not active as has been demonstrated for soils [12] and sediments [13] where most organisms demonstrate biomarkers of stationary growth phase and have accumulated PHA indicative of carbon accumulation without cell division [14].

### BRIEF DESCRIPTION OF THE FIGURES

The file of this patent contains at least one drawing executed in color. Copies of this patent or patent application publication with color drawings will be provided by the Patent and Trademark Office upon request and payment of the necessary fee.

FIG. 1 illustrates an exemplary “bio-trap” according to the subject invention.

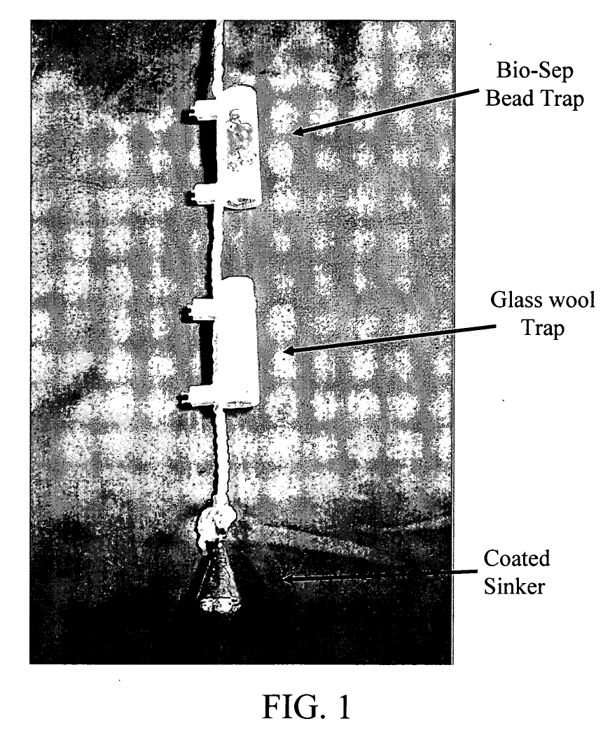

FIG. 2 depicts a laser confocal image (Live/Dead stain) of microbial colonization of a glass wool trap recovered from a perchloroethene (PCE) contaminated site. The image shows the glass wool strands and live (bright-green) bacterial colonies.

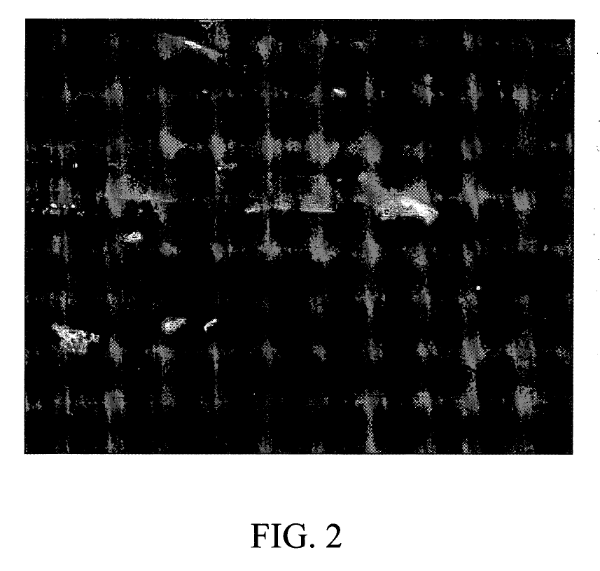

FIG. 3 is an image obtained by electron scanning microscopy of a BIO-SEP bead cross-section (FIG. 3A), its surface (FIG. 3B), and its colonization by bacteria (FIG. 3C).

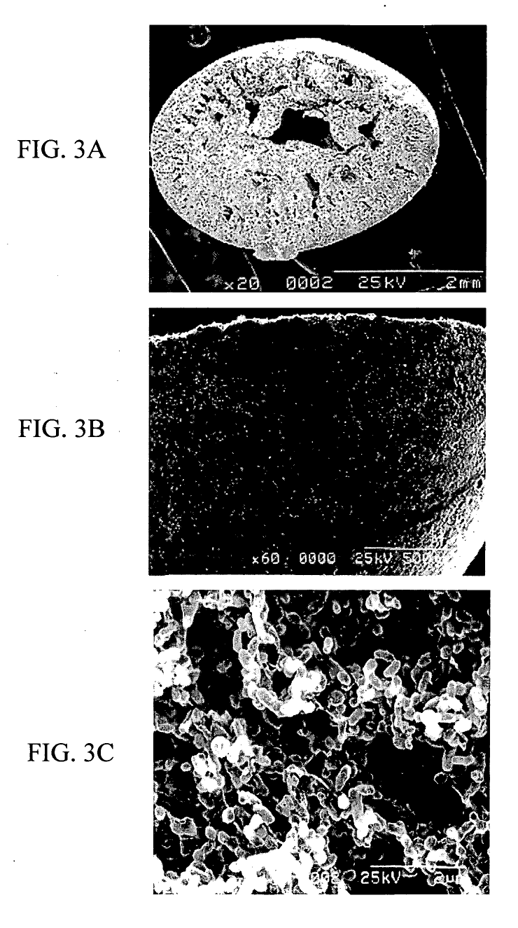

FIG. 4A: Confocal-laser scanning microscopy (CLSM) image of the cross section of Bio-Sep bead showing surface area for colonization; FIG. 4B: CLSM close-up of cross-section; FIG. 4C: CLSM of microbial colonization (Live/Dead stain) of bead recovered from a PCE contaminated site.

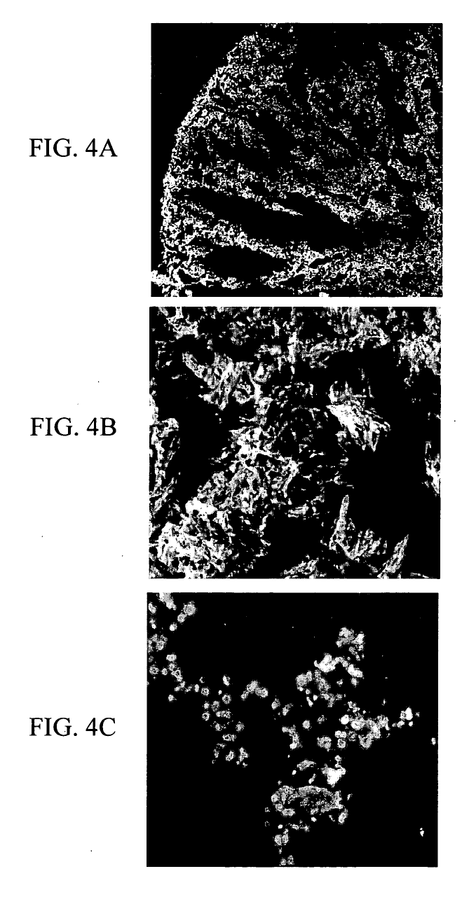

FIG. 5 is a scanning electron microscopy (SEM) image of microbial colonization of a biotrap solid support.

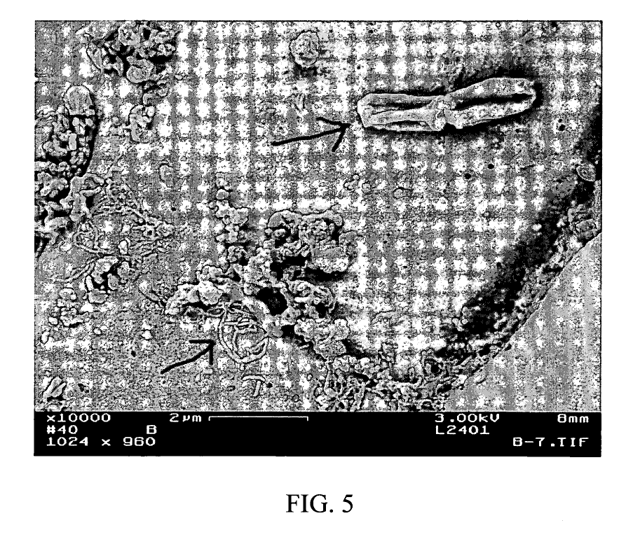

FIG. 6 shows a SEM image of microbial colonization of glass wool as a bio-trap solid support.

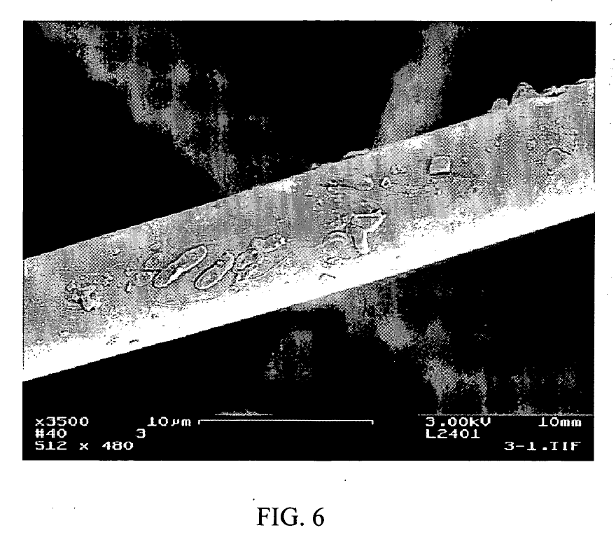

### BRIEF SUMMARY

In situ Monitored Natural Attenuation (MNA) or Active Bioremediation (Active Bio) is an attractive, cost-effective solution to the cleanup of many contaminated sites. We have developed a simple, but effective monitoring and test system to assess the practicality of in situ bio-remediation as a treatment option. Depending upon site characteristics, applications can include a multitude of approaches such as the addition of compounds to stimulate naturally occurring bioprocesses.

Documenting whether the desired bioprocesses are occurring or are likely to occur is critical to a defensible risk-based management approach for contaminated sites. Gathering the necessary evidence for these processes can be a costly undertaking. Currently, in situ geochemical parameters from a given site must be collected and analyzed over an extended period of time, and difficulties with data interpretation often occur. Such field data are usually coupled with expensive in vitro batch or column tests and the predominant bioprocesses are deduced. However, the definitive signature for bioremediation is in the microbial ecology of the site and the resulting biotransformation of contaminants. The subject invention provides methods for documenting whether the desired bioprocesses are occurring or are likely to occur at a given contaminated site by coupling the analytical power of lipid/DNA techniques with the use of simple in situ microbial sampling devices that also act as sensitive recorders of bioremediation.

Building on the use of automated accelerated solvent extraction techniques, we can now rapidly assess neutral and polar lipids. These new methods combined with GC and LC/MS/MS detection allow rapid analysis of biomarkers (such as and not limited to, respiratory quinones, diglycerides, sterols, intact phospholipids, poly beta-hydroxyalkonates, archaeol and caldarchaeols, ornithine lipids, sphingolipids, carotenoides, glycerides, glycolipids, gangliosides, eicosanoids, hopanes, isoprenoids, terpenes, fatty acids, fatty alcohols, waxes, fatty aldehydes, proteolipids and/or lysolipids) at greatly increased sensitivities and in a small fraction of the time required for classic solvent extraction. Coupled with selective DNA analysis, these tools can provide information regarding the microbial biomass, community structure, physiological state, and dominant redox processes at a contaminated site.

The subject invention also provides a systematic and uniform field approach to demonstrate which microbial bioprocesses are occurring, or may occur with certain manipulations in situ via the deployment of sampling devices (bio-traps). Bio-traps (essentially small in situ microcosms) can, in some embodiments, comprise sterilized glass wool or powder activated carbon (Bio-Sep®) beads in ⅝″×1″ polyflouryloxy (PFA) tubes attached to a nylon line or integrated with passive multilevel geochemical samples (MLS). Indigenous microbes colonize the traps during an in situ deployment period at the depth of the screened zone at a particular site (for example, in a monitoring well). After retrieval, the bio-trap contents are analyzed for biomarkers (such as lipids and/or DNA) that establish the effective in situ conditions that promote bio-remediation of a particular contaminant. Since the microbes integrate their responses over time, assurance is provided that the proper conditions are maintained within the site during the deployment. Additionally, non-radioactive labeled (¹³C) substrates or contaminants and/or surrogates may be added to the traps to stimulate a given response from the indigenous microbial community. These responses are directly measured via the cellular lipids with IR/MS and direct proof of occurrence of the predicted bioprocesses can be obtained. Finally, the content and state of the contaminants (parent and daughter products) in the trap can determined as a means of estimating the rate of biotransformation.

Bio-traps can be used to inexpensively assess the response of the subsurface microbial community to biostimulation, and may also be used for monitoring the in situ conditions. This is a readily deployable tool for directly determining the effectiveness of bioremediation at a specific site. Both reduction in the cost of site assessment and the more appropriate application of future pilot-scale biostimulation tests can lead to significant cost savings for those organizations involved in bioremediation.

### DETAILED DISCLOSURE

The subject invention provides a robust means for measurement of microbial metabolic activity that utilizes the incorporation of stable isotopes into cellular biomarkers. Stable isotope incorporation into microbial biomarkers is a well-established tool in microbial ecology [15]. Isotope incorporation into biomarkers is most effectively accomplished if solid phase samplers can be readily colonized by the resident microbiota. Thus, a solid phase sampler can be loaded with stable isotope enriched substrate and brought into contact with a microbial biofilm. The intimate contact of the isotope enriched substrate with the biofilm formed thereon results in the incorporation into the isotopes into biomarkers of specific components of the biofilm community.

The subject invention provides a single solvent-resistant perforated tube (or a plurality of connected solvent-resistant tubes) that contain a solid support on which biofilms can be formed. In certain embodiments, the tube(s) comprise autoclavable sterilizable/cleanable/solvent-resistant materials, including, but not limited to plastics, glass, ceramics, metals (such as, but not limited to, stainless steel, copper, or titanium), fluoro-polymers (such as, but not limited to, ethylene tetrafluoroethylene (ETFE), fluoriated ethylene propylene Teflon (FEP), tetrafluoroethylene Teflon (TFE), or polyfluoroalkoxy Teflon (PFA). The tube(s) can be of any pre-determined length. In certain preferred embodiments, PFA tubes about 1.5″ long and about ⅝″ in diameter are used. A perforated tube (or perforated tubes), according to the subject invention, can be stuffed with glass or metal beads, thin strands of glass or metal, glass wool (optionally, incinerated to remove organic carbon) and/or filled with Bio-Sep® beads. Where a plurality of perforated tubes is used in the practice of the invention, the tubes may be connected by a variety of means, including and not limited to, wire, rope, twine, nylon, solvent-resistant plastics, chain, metals (such a stainless steel, copper, or titanium), etc.). Connected tubes may be, optionally, weighted to assist in the deployment of the biotraps.

In other embodiments, biotraps can comprise a solid tube, such as a well casing, that contains one, or a plurality of separated, compartment(s) that are perforated. The compartment(s) can, optionally, be filled with a solid support. In various embodiments that utilize a plurality of separated compartments, certain selected compartments may be perforated at pre-selected intervals whereas other compartments are not perforated.

Bio-Sep® beads [described in U.S. Pat. No. 5,486,292, which is hereby incorporated by reference in its entirety] are 2-3 mm spherical beads consisting of 25% (w/w) aramid polymer (Nomex) and 75% (w/w) powdered activated carbon (PAC). The bulk density is about 0.16 g/cm³ with a porosity of 74%, and adsorptive capacity is greater than 600 m²/g. The beads are surrounded by an ultrafiltration-like membrane with a median pore diameter of 1.9 microns and with some large macropores>20 microns. Beads can be purged of organic carbon by incubation at 300° to 350° C. for at least 5 hours prior to deployment. The Nomex membrane resists formation of a surface biofilm with immobilization occurring through entrapment. Bacteria can be immobilized inside Bio-Sep® beads by culturing the bacteria in the presence of the beads if a carbon-absorbable species is a limiting nutrient and used to treat sulfide containing water [17]. The Biosep® beads can contain a variety of surfaces and can be spiked or coated with zero valence iron, manganese minerals, copper powders, sulfur granules, limestone powder, crushed silica. Other solid substrates (coupons) can be fabricated of magnetite, mica sheets, marble, glass, slow release polylactate polymers, or oxygen releasing chemicals.

Solid phase samplers with these beads have been shown to amplify formation of biofilms in drinking water. When compared to a biofilm generated on a plastic surface in the same water, the Bio-Sep® bead-generated, biofilm-viable biomass (as measured by PLFA) was about 23% in 1 day, 43% in 3 days and 130% in 14 days of that generated in a month on the plastic surface [16, 18A]. Similarly, experiments indicate the Bio-Sep® beads encouraged 10-times (˜50 nmoles phospholipid fatty acids/5 g trap) vs ˜1.3 nmoles PLFA/trap on the glass wool after incubation 1.5 months in a groundwater well field [18A, 18B, 19, 20].

Accordingly, a principal object of the present invention is to provide methods that quantitatively assess the specific metabolic activities in subsets of the microbial communities that are generated as biofilms on solid phase samplers designed to provide concentrations of stable isotope labeled substrates/nutrients for the subset to incorporate. The multi-species microbial community is incubated in the environment to be examined, recovered and biomarkers isolated for analysis of chemical composition and structure and the stable isotope abundance in specific biomarkers determined by isotope ratio mass spectrometry. From a series of solid phase samplers recovered over a time course, rates of isotope incorporation provide quantitative measures of rates at the in situ environmental conditions. The local environment of the solid phase samplers can be manipulated to modify the rates of the desired metabolic process measured by these methods by changing the pH of the local environment, changing redox levels, addition of specific substrates, addition of trace nutrients, etc. and, where permitted, via bio-augmentation (e.g., addition of bacteria or fungi).

In various embodiments, specific substrates can be used to stimulate specific groups or organisms. Hydrogen gas released directly or generated can be utilized by special bacteria but does not foster carbohydrate polymer slime formation that can plug aquifers; acetate utilized by a limited number of organisms as a major carbon source; methane used by a limited number of aerobic organisms, and only under special pressure and temperature conditions anaerobically. Many refractory chemicals such as poly aromatic hydrocarbons, tars, asphaltenes, chorobenzenes or polychlorobiphenyls are utililized by very selective group of organisms. Inorganic substrates such as sulfides are used by carbon dioxide fixing chemoautotrophs. Trace nutrients required are most often phosphate containing compounds or nitrogen containing compounds which may be added as supplements. Many organisms have other trace element needs that are usually met by multi-component mixes. Tungsten is required by some anaerobic archea and nickel by some methane-forming anaerobes.

In yet other embodiments, specific organisms with natural or engineered traits or genes are utilized to bioaugment special contaminant plumes. For example, Pseudomonas stutzeri KS secreting pyridine-2,6-bis(thiocarboxylic acid) (pdtca), a small secreted metabolite that has a high affinity for transition metals and increases iron uptake efficiency by 20% and has the ability to reduce both soluble and mineral forms of iron can be used. The copper complex of pdtca chemically destroys carbon tetrachloride. Another organism utilized widely in bioaugmentaion is Dehalococcoides ethenogenes that degrades trichlorethylene to ethane without forming vinyl chloride. Other organisms suitable for use in bio augmentation are well known to those skilled in the art.

In another aspect of the invention, it is possible to quantitatively measure metabolic rates based on incorporation of stable isotope labeled substrates (or the use of natural isotope abundance of substrates locally provided to solid phase samplers) directly measured in situ or in modified in situ conditions. Rates are measured from changes in isotope ratios in biomarkers recovered from the multispecies microbial communities colonizing the solid phase samplers. Biomarkers include, but are not limited to lipids, nucleic acids, proteins, carbohydrates that are sufficiently rare to be “signatures” characteristic of subsets of the multispecies microbial communities. Thus, the subject invention also provides a method for measuring rates of incorporation of stable non-radioactive isotopes into signature biomarkers of subsets of the multispecies microbial communities.

The subject invention also provides methods of identifying: 1) the microbial flora; or 2) the active bioremediation pathways at a site comprising:

a) contacting the microbial flora at the site with a solid support loaded or coated with a substrate that comprises an isotope;

b) incubating said solid support for a period of time sufficient to establish a biofilm on said solid support;

c) identifying biomarkers into which isotopes have be incorporated; and

d) correlating the biomarkers with particular microbes or components of a bioremediation pathway.

This invention provides quantitative analysis of the entire viable microbial community (which contain polar lipids), a means of identifying the community composition, and the nutritional/physiological status without the necessity of isolation and culture of the organisms [1, 4-6]. Recovery of nucleic acids can be facilitated by first extracting the lipids with the one-phase solvent extraction then recovering the nucleic acids [23]. Incorporation of stable isotopes into small subunit RNA of subsets of the multispecies microbial communities has also been demonstrated [24]. Specific oligonucleotides are attached via biotin-streptavidin to magnetic beads and rRNA capable of binding to the specific sequences on be beads recovered in a magnetic field, the RNA recovered and the ¹³C/¹²C ratio determined by isotope ratio MS. Another major biomarker, proteins, can be identified after their release from the cells by “top down” analysis in which known molecular weight components can be injected into quadrapole ion traps as multiply charged entities, the charges neutralized to a single charge within the trap and then specific molecular masses partially sequenced and enrichments detected [25].

In addition to the embodiments described throughout the subject application, the subject invention also provides the following non-limiting embodiments:

A(1). A quantitative method for detecting specific microbial activity comprising measuring the incorporation of stable isotopes in specific biomarkers by an active subset of microbes of a multi-species microbial community in an environment, wherein the microbes of a multi-species microbial community are brought into contact with at least one solid support (or a plurality of solid supports) capable of supporting the growth of a biofilm;

A(2). A method of assisting in situ bioremediation comprising:

a) deploying at least one biotrap (or a plurality of biotraps) at, or in a site or environment, said biotrap comprising a solid phase sampler containing one, or a plurality, of openings, said solid phase sampler optionally containing a solid support;

b) allowing said biotrap to remain at, or in, said site or environment for a period of time sufficient to allow said biotrap to become colonized by microbes indigenous to said site of environment;

c) retrieving or recovering said biotrap from said site or environment; and

d) analyzing the biotrap contents to determine those microbes that have colonized said biotrap; or

A(3). A method of obtaining a desired compound/substance, or degrading a desired compound/substance, comprising:

a) deploying at least one biotrap (or a plurality of biotraps) at, or in a site or environment, said biotrap comprising a solid phase sampler containing one, or a plurality, of openings, said solid phase sampler optionally containing a solid support that is supplemented with nutrients or other conditions that maximize the production and/or recovery of a desired compound or substance and/or wherein said nutrients preferentially select for the colonization of a biotrap with an organism capable of metabolizing or providing the desired compound or substance;

b) allowing said biotrap to remain at, or in, said site or environment for a period of time sufficient to allow said biotrap to become colonized by a desired microorganism or by organisms indigenous to said site or environment;

c) retrieving or recovering said biotrap from said site or environment; and

d) analyzing the biotrap contents to determine those microbes that have colonized said biotrap and/or to determine if a desired compound or substance has been produced; or

B. The method according to embodiments A1, A2, or A3, wherein the environment or site includes, and is not limited to, liquid, environmental, clinical, veterinary, agricultural, food, gaseous, liquid, and solid environmental matrices or samples. The method may also be used to test sites or environments such as “down-well” groundwater, various water bodies, microcosms, reactors, pipes, tanks, or vapor fields.

C. The method according to any preceding embodiment, wherein the method collects or concentrates microbes on all manner of solid phase sampler surfaces, such as glass wool and/or BIO Beads.

D. The method according to any preceding embodiment, further comprising the analysis or recovery of biomarkers from microbes that comprise a microbial community that has colonized the biotrap after incubation of a solid support in an environment or site, wherein the biomarkers contain (comprise) a stable isotope. Biomarkers include, and are not limited to, respiratory quinones, diglycerides, sterols, intact phospholipids, poly beta-hydroxyalkonates, archaeol and caldarchaeols, ornithine lipids, sphingolipids, carotenoides, glycerides, glycolipids, gangliosides, eicosanoids, hopanes, isoprenoids, terpenes, fatty acids, fatty alcohols, waxes, fatty aldehydes, proteolipids and/or lysolipids.

E. The method according to any preceding embodiment, wherein the solid support is a solid phase sampler (biotrap) that is colonized by in situ microorganisms.

F. The method according to any preceding embodiment, wherein the in situ microbiota can be augmented by addition of specific microbes, or enzymes in the solid support or solid phase samplers prior to environmental exposure.

G. The method according to any preceding embodiment, wherein the solid support or solid phase sampler comprises a coating or substrate that changes the pH, redox potential, adds trace nutrients (heavy metals, phosphate, nitrogen etc.) or other specific substrates to test the feasibility of manipulating the specific or general microbial activity under examination.

H. The method according to any preceding embodiment, wherein the stable isotope is selected from the group consisting of ²H, ¹³C, ¹⁵N, and those set forth in Table 1 (e.g., isotopes selected from the 225 available at Oak Ridge National Laboratory (Worldwide Website ornl.gov/isotopes or available via the United States Department of Energy “Isotope Production and Distribution Catalog”) including S, Ca, Fe).

I. The method according to any preceding embodiment, wherein the solid support or solid phase sampler contains a solid substrate that is coated with enriched substrates/nutrients (such as, but not limited to, acetate, lactate, amino acids, proteins, glucose, etc.) and which facilitate incorporation the material into biomarkers (cellular constituents unique enough to be associated with a specific group of microbes).

J. The method according to any preceding claim, wherein the stable isotope is naturally occurring and can be added to the solid support or solid phase samplers and be incorporated into biomarkers (cellular constituents unique enough to be associated with a specific group or microbes).

K. The method according to any preceding embodiment, wherein the recovery of biomarkers is performed by extraction of lipids with the one-phase solvent extraction or accelerated solvent extraction and nucleic acids and/or proteins are recovered from the aqueous portion of an extraction mixture.

L. The method according to any preceding embodiment, wherein the assessment of volatile and non volatile components is performed by fractionating lipids by silicic acid chromatography on the basis of polarity and/or separating free fatty acids, respiratory quinones, sterols, carotenoids, glycolipids, polar lipids, and intact lipids.

M. The method according to any preceding embodiment wherein the assessment of volatile and non-volatile components is conducted by capillary gas liquid chromatography.

N. The method according to any preceding embodiment, wherein the assessment of nonvolatile components is performed by high performance liquid chromatography/atmospheric pressure ionization/tandem mass spectrometry.

O. The method according to any preceding embodiment, wherein the assessment of isotope enrichment in biomarkers in potentially volatile components is conducted by gas chromatography isotope ratio mass spectrometry.

P. The method according to any preceding embodiment, wherein the assessment of isotope enrichment in biomarkers is conducted by pyrolysis (with or without in situ derivatization and isotope ratio mass spectrometry).

Q. The method according to any preceding claim, wherein the assessment of isotope enrichment in rRNA, mRNA, DNA, is performed by a means that includes, but is not limited to, concentration with oligonucleotide bound magnetic beads or density gradient centrifugation followed by isotope ratio mass spectrometry.

R. The method according to any preceding embodiment, wherein the assessment of isotope enrichment in proteins is performed by the following non-limiting means: concentration with immunologic affinity surfaces and assessment with “top down” electrospray external ionization, detection by ion-trap mass spectrometry utilizing (MS)ⁿ after “ion parking” with detection/identification isotope ratio mass spectrometry.

S. The method according to any preceding embodiment, wherein the isotope is a stable isotope that is, optionally, non-radioactive.

T. The method according to any preceding embodiment, wherein said desired compound or substance: a) is a break-down product of a contaminant suitable for bioremediation; b) is a metal; or c) is precipitated.

### EXAMPLE 1

### Demonstration of the Utility of the Proposed Process in Defining a Subset of the Multispecies Microbial Communities Colonizing the Solid Phase Samplers:

Down well solid phase samplers were loaded with ¹³C-enriched benzene and incubated for a month in a BTEX contaminated groundwater site. On recovery, a minor portion of specific PLFA biomarkers from the biofilm showed significantly increased ¹³C levels after analysis by gas chromatography/isotope ratio mass spectrometry. In these experiments about 80% of the ¹³C-labeled benzene was degraded with essentially no exchange with the benzene in the aquifer and both 16 and 18 carbon saturated and monounsaturated as well as iso and anti-iso branched saturated PLFA were shown to have incorporated ¹³C after recovery from the Bio Sep beads incubated in the contaminant plume (R. Geyer, A. Peacock and M. Kastner [UFZ Leipzig-Halle, Germany]). This experiment establishes several critical points:

1. Active microbial biofilms form in solid phase samplers;

2. Solid phase samplers can concentrate stable isotope labeled substrates sufficiently well and for a long enough time that microorganisms in the bead surface biofilm can incorporate them into biomarkers; and

3. Extraction of biomarkers, derivatization and GC/IRMS analysis of the biomarkers of the multi-species biofilm microbial community shows only a small subset were actively incorporating the ¹³C establishing their metabolic activity.

### EXAMPLE 2

### Manipulation of Microbial Communities

If the aim of a microbial community manipulation is to bio-immobilize a desired compound, such as uranium (U) in a subsurface plume by the metabolic reduction of U(VI) to U(IV) by an organism such as Geobacter, then ¹³C-acetate incorporation should parallel the accumulation of U(IV). The subject invention provides methods for the examination of such processes in situ (e.g., in the location of an U deposit). Solid phase samplers or solid supports are suspended in the plumes in wells, and substrates are provided that provide the necessary nutrients and redox conditions and other features to maximize recovery of U(IV) in the recovered solid phase extractors or solid supports. Infusion of 1-3 mM acetate solid supports or solid phase samplers inserted into the subsurface at Rifle, Colo., UMTRA site induced a greater than 3-fold increase in viable biomass, with a 7 to 10-fold increase in the specific PLFA/plasmalogens characteristic of Geobacter and low-G+C Gram-positive Clostridium, Lactosphaera, Desulfosporosinus, dissimilatory Fe(III) reducers. PLFA biomarkers were increased as compared to upgradient sites and local microbial community metabolic activity parameters in down-hole microcosm solid phase “bio-traps” with BioSep® beads better predict efficacy of bioimmobilization than viable biomass and community composition matches to the groundwater and/or sediment microbial communities.

The capacity of the solid phase sampler system to document the correlation of metal precipitation with specific microbial metabolism has also been demonstrated with uranium in a laboratory reconstruction experiment. Geobacter sulforeducens in modified artificial groundwater supplemented with 10 mM ¹³C-acetate and 200 ppm UCl₄ formed a biofilm on silicon wafers in 2 days at 30° C. Micro and nano-SIMS (secondary ion mass spectrometry) shows high congruence between uranium oxide deposition and the bacteria that formed a biofilm containing ¹³C-lipids and ¹³C-macromolecules [21,22] Electron dispersive spectroscopy (EDX) was also used to chemically characterize the surface of the biofilm and locate the uranium-rich regions which were congruent with the bacteria. Bright-field scanning transmission electron microscopy (BF-STEM) was used to obtain distribution maps of number of cations including U at the nano-scale and show congruency between U and O. High-resolution images of the surface using high-angle annular dark field scanning electron microscopy (HAAD-STEM) reveal that these biominerals occur as patches on the surface of the bacteria and were precipitated as uranium oxide minerals. Selected area electron diffraction (SAED) patterns of these patches were acquired so that the structure of the uranium nano-biominerals can be inferred. The SEAD patterns of the uranium biominerals indicate that the uranium oxide minerals are largely UO_{2.87} and, therefore, are stable over a range of redox conditions.

### EXAMPLE 3

### Down-Well Microcosm “Bio-traps” and Subsurface Sediments for Rapid Expanded-Lipid-Biomarker Analysis and DNA Recovery for Monitoring Bioremediation Microbial-Community Ecology Within Samples from Uranium-Contaminated Sites

Microbial activity is of primary importance in the bioremediation of metal-contaminated subsurface environments. One of our principal objectives is to develop more expedient and cost-effective methods for biomarker recovery and analysis, utilizing a combination of biomarkers (including and not limited to: expanded signature lipid biomarkers (SLB), polymerase chain reaction (PCR) denaturing gradient gel electrophoresis (DGGE), terminal restriction fragment length polymorphism (T-RFLP) analyses of 16S rDNA, and specific genes). These tools and the information provided thereby are used to define the viable community composition and provide indications of important specific activities relative to the impact of metals and radionuclides on indigenous microbial communities.

Emplacement of sterile surfaces, “Bio-traps”, (with and without nutrient sources) in the well fields of impacted sites was performed. After a 2-3 week exposure, the traps were recovered and analyzed. Traditionally, methods employed to monitor microorganisms require ex situ culture analysis of groundwater membrane retentates and sediments. However, these methods poorly represent in situ microbial communities. Phospholipid fatty acid (PLFA) analysis has been utilized to determine shifts in microbial biomass, nutritional/physiological status, and community diversity in situ. For greater specificity, we have complemented PLFA analysis with a PCR-DGGE approach, employing primers that recognize the 16S rDNA of almost all known and inferred bacterial species and of specific functional genes. Sequence analysis of individual bands from DGGE gels was used to provide fine-scale biomarkers and loosely infer the identity of the source organisms, using database searches and phylogenetic methods related to the complexity, band positions, and relative band intensities of DGGE patterns to contaminant load. Unfortunately, these monitoring technologies require at least several days of intensive work with highly skilled personnel. To exploit potential economies in sampling realized by the use of the “Bio-traps,” we developed methods that are faster and require less specialized analyses, yet provide more comprehensive insight into the in situ microbial ecology.

Room-temperature extraction, fractionation, derivatization, and GC/MS analysis for PLFA requires at least three working days. We have shown that high-temperature/high-pressure sequential extraction can be completed in less than an hour. The lipid-extracted residue can be acid-hydrolyzed with “magic” methanol (“magic” methanol is anhydrous methanol:chloroform:concentrated HCl:10:1:0.5, v/v), and the ester linked hydroxy (OH) fatty acids of the lipopolysaccharide are readily detected by gas chromatography/mass spectrometry (GC/MS). Utilization of high performance/atmospheric pressure chemical ionization/tandem mass spectrometry (HPLC/APCI/MS/MS), particularly of the ubiquinones (UQ), menaquinones (MK), and intact phospholipids, expands insight into metabolic activities and increases sensitivity several fold. T-RFLP expands the purview of DNA analyses from DGGE without a great increase in time. The liquid chromatography/tandem mass spectrometry (LC/MS/MS) analysis of UQ/MK allow monitoring manipulations of in situ terminal-electron-acceptor concentration that are critical to heavy-metal and radionuclide immobilization.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

The following references are hereby incorporated by reference in their entireties:

[1] Pinkart, H. C., D. B. Ringelberg, Y. M. Piceno, S. J. Macnaughton, and D. C. White. 2002. Biochemical approaches to biomass measurements and community structure analysis. In Manual of Environmental Microbiology, 2^{nd} Edition (D. E. Stahl, C. H. Hurst, G. R. Knudsen, M. J. McInerney, L. D. Stetzenbach, and M. V. Walter, eds.) American Society for Microbiology Press, Washington, D.C. pp. 101-113.

[2] White, D. C., W. M. Davis, J. S. Nickels, J. D. King and R. J. Bobbie. 1979. Determination of the sedimentary microbial biomass by extractable lipid phosphate. Oecologia 40: 51-62.

[3] White, D. C., R. J. Palmer, R. S. Burkhalter, S. J. Macnaughton, J. R. Stephen, R. G. Kern, K. J. Venkateswaran, C. A. Smith, Y-D. Gan, Y-J. Chang, S. L. Whitaker, and R. J. Moss. 1999. Utilization of signature biomarkers to define microbial communities in biofilms and as contaminants of spacecraft searching for extraterrestrial life. Second COST 520 Workshop, Biofouling and Materials, Ecole d'Ingenieurs du Valais, Sion Switzerland (J. Weber and W. Sand, Eds., EDMZ, Bern Switzerland) pp. 41-62, June 9-13.

[4] White, D. C., J. O. Stair, and D. B. Ringelberg. 1996. Quantitative Comparisons of in situ Microbial Biodiversity by Signature Biomarker Analysis. J. Indust. Microbiol. 17: 185-196

[5] White, D. C., C. A. Lytle, Y-D. M. Gan, Y. M. Piceno, M. H. Wimpee, A. Peacock and C. A. Smith 2002. Flash Detection/identification of Pathogens, Bacterial Spores and Bioterrorism Agents Biomarkers from Clinical and Environmental Matrices. J. Microbial Methods 48: 139-147.

[6] Stephen, J. R., Y-J. Chang, Y. D. Gan, A. Peacock, S. M. Pfiffner, M. J. Barcelona, D. C. White, and S. J. Macnaughton. 1999. Microbial Characterization of JP-4 fuel contaminated-site using a combined lipid biomarker/PCR-DGGE based approach. Environmental Microbiology. 1: 231-241.

[7] Ivanova, I A., J. R. Stephen, Y-J. Chang, , J. Bruggemann, P. E. Long, J. P. McKinley, G. A. Kowalchuk, D. C. White, and S. J. Macnaughton. 2000. A survey of 16S rRNA and amoA genes related to autotrophic ammonia-oxidizing bacteria of the β-subdivision of the class proteobacteria in contaminated groundwater. Canad J. Microbiol. 46: 1012-1020.

[8] Chang, Y-J. A. Peacock, P. E. Long, J. R. Stephen, J. P. McKinley, S. J. Macnaughton, A. K. M. Anwar Hussain, A. M. Saxton, and D. C. White. 2001. Diversity and Characterization of Sulfate-Reducing Bacteria in Groundwater at a Uranium Mill Tailings Site. Appl. Environ. Microbiol. 67: 3149-3160.

[9] White, D. C. 1995. Chemical ecology: Possible linkage between macro-and microbial ecology. Oikos 74: 174-181.

[10] Lytle, C. A., Y-D. M. Gan, K. Salone, and D. C. White 2001. Sensitive Characterization of Microbial Ubiquinones from Biofilms by Electrospray/Mass Spectrometry Environ. Microbiol. 3 (4): 265-272.

[11] White, D. C., D. B. Ringelberg, and S. J. Macnaughton. 1997. Review of PHA and signature lipid biomarker analysis for quantitative assessment of in situ environmental microbial ecology. In 1996 International Symposium on Bacterial Polyhydroxylalkanoates, (G. Eggink, A. Steinbuchel, Y. Poirer, and B Witholt, eds.) NRC Research Press, Ottawa, Canada, pp. 161-170

[12] Kieft, T. L., D. B. Ringelberg, and D. C. White. 1994. Changes in ester-linked phospholipid fatty acid profiles of subsurface bacteria during starvation and desiccation in a porous medium. Appl. Environ. Microbiol. 60: 3292-3299.

[13] Findlay, R. H., P. C. Pollard, D. J. W. Moriarty, and D. C. White. 1985. Quantitative determination of microbial activity and community nutritional status in estuarine sediments: evidence for a disturbance artifact. Canad. J. Microbiol. 31: 493-498.

[14] Tunlid, A., B. H. Baird, M. B. Trexler, S. Olsson, R. H. Findlay, G. Odham, and D. C. White. 1985. Determination of phospholipid ester-linked fatty acids and poly beta hydroxybutyrate for the estimation of bacterial biomass and activity in the rhizosphere of the rape plant Brassica napus (L.). Canad. J. Microbiol. 31: 1113-1119.

[15] Boschker, H. T. S., and J. J. Middelburg 2002. Stable isotopes and biomarkers in microbial ecology. FEMS Microbiology Ecology 40 85-95.

[16] White, D. C., J. S. Gouffon, A. D. Peacock, R. Geyer, A. Biemacki, G. A. Davis, M. Pryor, M. B. Tabacco, and K. L. Sublette. 2003. Forensic Analysis by Comprehensive Rapid Detection of Pathogens and Contamination Concentrated in Biofilms in Drinking Water Systems for Water Resource Protection and Management, Environmental Forensics (accepted January 2003).

[17] Sublette, K. L., A. E. Plato, M. Woolsey, R. G. Yates, C. E. Camp, and T. Bair. 1996. Immobilization of a Sulfide-oxidizing Bacterium in a Novel Adsorbent Biocatalyst Support”, Applied Biochem. Biotech., 57/58, 1013-1019.

[18A] Gouffon, J. S., R. Geyer, A. D. Peacock, Y-D. Gan, Y-J Chang, K. Salone, C. Lytle, K. L. Sublette, and D. C. White. 2002. Rapid Quantitative Detection of Pathogens & Contamination by Analysis of Biofilms Generated on Coupons in Water Resource Management. Proceedings of the Third International Conference on Water Resources and Environmental Research (ICWRER) (edit. by G. H. Schmitz). Dresden Germany, July 22-25, Eigenvertag des Forum fur Abfallwirtschaft und Altlasten eV, Pirna D-01796 Germany, Volume II, Pp. 305-310.

[18B] Peacock A. D. , Y-J. Chang, J. D. Istok and D. C. White. 2003. Utilization of Microbial Biofilms as Monitors of Bioremediation. Microbal Ecology (P. Hirsch festschrift issue). July 2003.

[19] Sublette, K. L., A. D. Peacock, G. A. Davis, M. C. Harrison, R. Geyer and D. C. White 2002. Convenient, down-well, in-situ monitoring of chlorinated hydrocarbon remediation with sterilizable “bug traps” containing Bio Sep® beads, Abstract 171, page 46. at the 2002 International Society for Subsurface Microbiology, Copenhagen Denmark.

[20] Peacock, A D., R. T. Anderson, Y-J. Chang, P. E. Long, and D. C. White. 2002. Biostimulation of Metal-Reducing Microbes at a Former Uranium Mill Tailings Site. Eos Trans. AGU, 83(47), Fall Meet. Suppl., Abstract B51B-0716 INVITED, 2002Abs AGU December meeting.

[21] White, D. C., M. Fayek, J. S. Gouffon, A. D. Peacock, S. M. Pfiffner, R. Geyer, and K. L. Sublette 2002. “Facile quantitative detection of microbial biofilms capable of potential in situ bioimmobilization of Uranium in aquifers. 2002 International Society for Subsurface Microbiology, Abstract, September 14, Copenhagen Denmark

[22] Fayek, M. J., S. Utsunomiya, S. M. Pfiffner, L. Anovitz, Y. A. Gorby, D. C. White, L. R. Riciputi, R. C. Ewing, & F. J. Stadermann. 2003. Predicting the Stability of Nano-scale Bio-precipitated Uranium Phases. Nature submitted.

[23] Kehrmeyer, S. R., B. M. Applegate, H. Pinkart, D. B. Hedrick, D. C. White and G. S. Sayler. 1996. Combined lipid/DNA extraction method for environmental samples, J. Microbiological Methods 25: 153-163.

[24] MacGregor, B. J., V. Bruchert, S. Fleischer and R. Aman. 2002. Isolation of small-subunit rRNA for stable isotope characterization. Environmental Microbiology 4(8): 451-464.

[25] McLusky, S. A., G. R. Reid, and J. M. Wells. 2002. Ion parking during ion/ion reactions in electrodynamic traps. Ann Chem. 74: 336-346.

TABLE 1
Isotopes

Actinium-225
Aluminum-26
Americium-241
Americium-243
Antimony
Argon (Alt)
Arsenic-72
Arsenic-73
Barium
Berkelium-249
Beryllium-7
Bismuth-207
Bromine
Bromine (Alt)
Cadmium
Cadmium-109
Calcium
Californium-249
Californium-252
Carbon (Alt)
Cerium
Chlorine
Chlorine (Alt)
Chromium
Cobalt-60
Copper
Copper-67
Curium-244
Curium-248
Dysprosium
Dysprosium-166
Erbium
Europium
Gadolinium
Gadolinium-148
Gallium
Germanium
Germanium-68
Gold-199
Hafnium
Helium (Alt)
Holmium-166
Holmium-166m
Indium
Iridium
Iridium-192
Iron
Iron-52
Iron-55
Krypton (Alt)
Lanthanum
Lead
Lithium
Lutetium
Magnesium
Magnesium-28
Mercury
Molybdenum
Neodymium
Neon (Alt)
Nickel
Nitrogen (Alt)
Osmium
Oxygen (Alt)
Palladium
Platinum
Plutonium-238
Plutonium-239
Plutonium-240
Plutonium-241
Plutonium-242
Polonium-209
Potassium
Radium-225
Rhenium
Rhenium-186
Rubidium
Rubidium-83
Ruthenium
Ruthenium-97
Samarium
Samarium-153
Selenium
Selenium-72
Selenium-75
Silicon
Silicon-32
Silver
Sodium-22
Strontium
Strontium-82
Strontium-85
Strontium-89
Sulfur
Sulfur (Alt)
Tantalum
Technetium-95m
Technetium-96
Technetium-99
Tellurium
Tellurium-123m
Thallium
Thorium-228
Thorium-230
Tin
Tin-117m
Titanium
Titanium-44
Tungsten
Tungsten-188
Tungsten-188 G
Uranium-234
Uranium-235
Uranium-238
Vanadium
Vanadium-48
Vanadium-49
Xenon (Alt)
Xenon-127
Ytterbium
Yttrium-88
Zinc
Zinc-65
Zirconium
Zirconium-88

1. A method of identifying active bioremediation pathways at a site comprising:
a) contacting the microbial flora at the site with a solid support loaded or coated with a substrate that comprises an isotope;
b) incubating said solid support for a period of time sufficient to establish a biofilm on said solid support;
c) identifying biomarkers into which isotopes have be incorporated; and
d) correlating the biomarkers with particular microbes or components of a bioremediation pathway.

2. The method according to claim 1, wherein said biomarkers are selected from the group consisting of lipids, nucleic acids, proteins, and carbohydrates.

3. The method according to claim 1, wherein said biomarkers are respiratory quinones, diglycerides, sterols, intact phospholipids, poly beta-hydroxyalkonates, archaeol and caldarchaeols, ornithine lipids, sphingolipids, carotenoides, glycerides, glycolipids, gangliosides, eicosanoids, hopanes, isoprenoids, terpenes, fatty acids, fatty alcohols, waxes, fatty aldehydes, proteolipids or lysolipids

4. The method according to claim 2, wherein said biomarkers are characteristic of a subset or microbial organisms.

5. The method according to claim 3, wherein said biomarkers are characteristic of a subset or microbial organisms.

6. The method according to claim 1, wherein said isotope is ²H, ¹³C, ¹⁵N, an isotope as set forth in Table 1 or a naturally occurring isotope.

7. The method according to claim 1, wherein said site is selected from the group consisting of: liquid samples; environmental samples; clinical samples; veterinary samples; agricultural samples; food samples; gaseous samples; liquid samples; solid environmental matrices or samples; “down-well” groundwater, various water bodies, microcosms, reactors, pipes, tanks, subsurface sites, and vapor fields.

8. The method according to claim 1, wherein the biomarkers are identified by one or more of the following methods: pyrolysis, optionally with in situ derivatization and isotope ratio mass spectrometry; phospholipids fatty acid (PFLA) analysis, polymerase chain reaction denaturing gradient gel electrophoresis (PCR-DGGE)., expanded signature lipid biomarker analysis (SLB), terminal restriction fragment length polymorphism (T-RFLP) analysis of 16S rDNA or specific genes, high performance/atmospheric pressure chemical ionization/tandem mass spectrometry (HPLC/APCI/MS/MS) or liquid chromatography/tandem mass spectrometry (LC/MS/MS).

9. A method of identifying the microbial flora at a site comprising:
a) contacting the microbial flora at the site with a solid support loaded or coated with a substrate that comprises an isotope;
b) incubating said solid support for a period of time sufficient to establish a biofilm on said solid support;
c) identifying biomarkers into which isotopes have be incorporated; and
d) correlating the biomarkers with particular microbes or components of a bioremediation pathway.

10. The method according to claim 9, wherein said biomarkers are selected from the group consisting of lipids, nucleic acids, proteins, and carbohydrates.

11. The method according to claim 9, wherein said biomarkers are respiratory quinones, diglycerides, sterols, intact phospholipids, poly beta-hydroxyalkonates, archaeol and caldarchaeols, ornithine lipids, sphingolipids, carotenoides, glycerides, glycolipids, gangliosides, eicosanoids, hopanes, isoprenoids, terpenes, fatty acids, fatty alcohols, waxes, fatty aldehydes, proteolipids or lysolipids

12. The method according to claim 10, wherein said biomarkers are characteristic of a subset or microbial organisms.

13. The method according to claim 11, wherein said biomarkers are characteristic of a subset or microbial organisms.

14. The method according to claim 9, wherein said isotope is ²H, ¹³C, ¹⁵N, an isotope as set forth in Table 1 or a naturally occurring isotope.

15. The method according to claim 9 wherein said site is selected from the group consisting of: liquid samples; environmental samples; clinical samples; veterinary samples; agricultural samples; food samples; gaseous samples; liquid samples; solid environmental matrices or samples; “down-well” groundwater, various water bodies, microcosms, reactors, pipes, tanks, subsurface sites, and vapor fields.

16. The method according to claim 9 wherein the biomarkers are identified by one or more of the following methods: pyrolysis, optionally with in situ derivatization and isotope ratio mass spectrometry; phospholipids fatty acid (PFLA) analysis, polymerase chain reaction denaturing gradient gel electrophoresis (PCR-DGGE)., expanded signature lipid biomarker analysis (SLB), terminal restriction fragment length polymorphism (T-RFLP) analysis of 16S rDNA or specific genes, high performance/atmospheric pressure chemical ionization/tandem mass spectrometry (HPLC/APCI/MS/MS) or liquid chromatography/tandem mass spectrometry (LC/MS/MS).
